(19) United States
(12) Patent Application Publication
(10) Pub. No.: US 2009/0030276 A1
(43) Pub. Date: Jan. 29, 2009

(54) TISSUE VISUALIZATION CATHETER WITH IMAGING SYSTEMS INTEGRATION

(75) Inventors: Vahid SAADAT, Atherton, CA (US); Ruey-Feng PEH, Mountain View, CA (US); Zachary J. MALCHANO, San Francisco, CA (US)

Correspondence Address: LEVINE BAGADE HAN LLP, 2483 EAST BAYSHORE ROAD, SUITE 100, PALO ALTO, CA, 94303, US

(73) Assignee: Voyage Medical, Inc., Campbell, CA (US)

(21) Appl. No.: 12/180,376

(22) Filed: Jul. 25, 2008

Related U.S. Application Data
(60) Provisional application No. 60/952,476, filed on Jul. 27, 2007.

(51) Int. Cl. A61B 1/04

(52) U.S. Cl. 600/112; 600/109

(57) ABSTRACT

Tissue visualization catheters with imaging systems integrated within the imaging catheter system are described. The tissue-imaging apparatus relates to devices and/or methods to provide visualization of tissue regions within a body lumen such as a heart, which is filled with blood flowing dynamically therethrough. High-resolution images can be obtained by miniaturizing and integrating solid state cameras into the tissue visualization catheter in a number of different off-axis configurations. One or more light sources can also be optionally integrated with the solid state imagers to illuminate the tissue from different angles.

### CROSS-REFERENCE TO RELATED APPLICATIONS

This application claims the benefit of priority to U.S. Prov. Pat. App. 60/952,476 filed Jul. 27, 2007, which is incorporated herein by reference in its entirety.

### FIELD OF THE INVENTION

The present invention relates generally to medical devices used for accessing, visualizing, and/or treating regions of tissue within a body. More particularly, the present invention relates to methods and apparatus for integrating solid state camera systems, such as CMOS imaging systems, into an imaging system to visualize tissue.

### BACKGROUND OF THE INVENTION

Conventional devices for visualizing interior regions of a body lumen are known. For example, ultrasound devices have been used to produce images from within a body in vivo. Ultrasound has been used both with and without contrast agents, which typically enhance ultrasound-derived images.

Other conventional methods have utilized catheters or probes having position sensors deployed within the body lumen, such as the interior of a cardiac chamber. These types of positional sensors are typically used to determine the movement of a cardiac tissue surface or the electrical activity within the cardiac tissue. When a sufficient number of points have been sampled by the sensors, a “map” of the cardiac tissue may be generated.

Another conventional device utilizes an inflatable balloon which is typically introduced intravascularly in a deflated state and then inflated against the tissue region to be examined. Imaging is typically accomplished by an optical fiber or other apparatus such as electronic chips for viewing the tissue through the membrane(s) of the inflated balloon. Moreover, the balloon must generally be inflated for imaging. Other conventional balloons utilize a cavity or depression formed at a distal end of the inflated balloon. This cavity or depression is pressed against the tissue to be examined and is flushed with a clear fluid to provide a clear pathway through the blood.

However, such imaging balloons have many inherent disadvantages. For instance, such balloons generally require that the balloon be inflated to a relatively large size which may undesirably displace surrounding tissue and interfere with fine positioning of the imaging system against the tissue. Moreover, the working area created by such inflatable balloons are generally cramped and limited in size. Furthermore, inflated balloons may be susceptible to pressure changes in the surrounding fluid. For example, if the environment surrounding the inflated balloon undergoes pressure changes, e.g., during systolic and diastolic pressure cycles in a beating heart, the constant pressure change may affect the inflated balloon volume and its positioning to produce unsteady or undesirable conditions for optimal tissue imaging. Additionally, imaging balloons are subject to producing poor or blurred tissue images if the balloon is not firmly pressed against the tissue surface because of intervening blood between the balloon and tissue.

Accordingly, these types of imaging modalities are generally unable to provide desirable images useful for sufficient diagnosis and therapy of the endoluminal structure, due in part to factors such as dynamic forces generated by the natural movement of the heart. Moreover, anatomic structures within the body can occlude or obstruct the image acquisition process. Also, the presence and movement of opaque bodily fluids such as blood generally make in vivo imaging of tissue regions within the heart difficult.

Other external imaging modalities are also conventionally utilized. For example, computed tomography (CT) and magnetic resonance imaging (MRI) are typical modalities which are widely used to obtain images of body lumens such as the interior chambers of the heart. However, such imaging modalities fail to provide real-time imaging for intra-operative therapeutic procedures. Fluoroscopic imaging, for instance, is widely used to identify anatomic landmarks within the heart and other regions of the body. However, fluoroscopy fails to provide an accurate image of the tissue quality or surface and also fails to provide for instrumentation for performing tissue manipulation or other therapeutic procedures upon the visualized tissue regions. In addition, fluoroscopy provides a shadow of the intervening tissue onto a plate or sensor when it may be desirable to view the intraluminal surface of the tissue to diagnose pathologies or to perform some form of therapy on it.

Thus, a tissue imaging system which is able to provide real-time in vivo images of tissue regions within body lumens such as the heart through opaque media such as blood and which also provide instruments for therapeutic procedures upon the visualized tissue are desirable.

### SUMMARY OF THE INVENTION

A tissue imaging and manipulation apparatus that may be utilized for procedures within a body lumen, such as the heart, in which visualization of the surrounding tissue is made difficult, if not impossible, by medium contained within the lumen such as blood, is described below. Generally, such a tissue imaging and manipulation apparatus comprises an optional delivery catheter or sheath through which a deployment catheter and imaging hood may be advanced for placement against or adjacent to the tissue to be imaged.

The deployment catheter may define a fluid delivery lumen therethrough as well as an imaging lumen within which an optical imaging fiber or assembly may be disposed for imaging tissue. When deployed, the imaging hood may be expanded into any number of shapes, e.g., cylindrical, conical as shown, semi-spherical, etc., provided that an open area or field is defined by the imaging hood. The open area is the area within which the tissue region of interest may be imaged. The imaging hood may also define an atraumatic contact lip or edge for placement or abutment against the tissue region of interest. Moreover, the distal end of the deployment catheter or separate manipulatable catheters may be articulated through various controlling mechanisms such as push-pull wires manually or via computer control

The deployment catheter may also be stabilized relative to the tissue surface through various methods. For instance, inflatable stabilizing balloons positioned along a length of the catheter may be utilized, or tissue engagement anchors may be passed through or along the deployment catheter for temporary engagement of the underlying tissue.

In operation, after the imaging hood has been deployed, fluid may be pumped at a positive pressure through the fluid delivery lumen until the fluid fills the open area completely and displaces any blood from within the open area. The fluid may comprise any biocompatible fluid, e.g., saline, water, plasma, Fluorinert™, etc., which is sufficiently transparent to allow for relatively undistorted visualization through the fluid. The fluid may be pumped continuously or intermittently to allow for image capture by an optional processor which may be in communication with the assembly.

In an exemplary variation for imaging tissue surfaces within a heart chamber containing blood, the tissue imaging and treatment system may generally comprise a catheter body having a lumen defined therethrough, a visualization element disposed adjacent the catheter body, the visualization element having a field of view, a transparent fluid source in fluid communication with the lumen, and a barrier or membrane extendable from the catheter body to localize, between the visualization element and the field of view, displacement of blood by transparent fluid that flows from the lumen, and an instrument translatable through the displaced blood for performing any number of treatments upon the tissue surface within the field of view. The imaging hood may be formed into any number of configurations and the imaging assembly may also be utilized with any number of therapeutic tools which may be deployed through the deployment catheter.

More particularly in certain variations, the tissue visualization system may comprise components including the imaging hood, where the hood may further include a membrane having a main aperture and additional optional openings disposed over the distal end of the hood. An introducer sheath or the deployment catheter upon which the imaging hood is disposed may further comprise a steerable segment made of multiple adjacent links which are pivotably connected to one another and which may be articulated within a single plane or multiple planes. The deployment catheter itself may be comprised of a multiple lumen extrusion, such as a four-lumen catheter extrusion, which is reinforced with braided stainless steel fibers to provide structural support. The proximal end of the catheter may be coupled to a handle for manipulation and articulation of the system.

To provide visualization, an imaging element such as a fiberscope or electronic imager such as a solid state camera, e.g., CCD or CMOS, may be mounted, e.g., on a shape memory wire, and positioned within or along the hood interior. A fluid reservoir and/or pump (e.g., syringe, pressurized intravenous bag, etc.) may be fluidly coupled to the proximal end of the catheter to hold the translucent fluid such as saline or contrast medium as well as for providing the pressure to inject the fluid into the imaging hood.

In an exemplary variation for imaging tissue surfaces, this application further shows various embodiments of the tissue visualization catheter with high resolution miniature imaging systems integrated into or along the imaging hood. Such miniature imaging systems can comprise solid state cameras enabled by CMOS (complementary metal oxide semi-conductors) or CCD (charged couple devices) technology where elements of the imaging device may be separated from one another and inter-connected by appropriate cable(s). Integrating the solid state cameras within or along the imaging hood allows for the imagers to be compacted into a small volume having a low profile and/or a flexible configuration.

Miniature imaging systems integrated within the tissue visualization catheter may be also arranged to provide off-axis visualization of imaged tissue surfaces distal to the imaging hood when opaque bodily fluids such as blood are purged from the hood. As compared to axially-oriented visualization along the axis of the catheter (for example, optical fibers positioned within a working channel of a catheter), off-axis visualization utilizing integrated solid state imagers may be particularly advantageous in allowing operators to view and gauge distances between deployed tools and tissue. Given the low-profile configuration of the imaging assembly, any number of instruments may be passed within the visual field of the hood for treating the underlying tissue. Off-axis visualization may also be particularly advantageous in providing visual confirmation of instrument-to-tissue contact as compared to axially-oriented visualization.

One or more light sources, e.g., light emitting diodes (LEDs), may also be surface mounted along the imaging assembly or separately in any number of configurations. For example, LEDs may be positioned circumferentially around the interior of the imaging hood on one or more hood support struts to provide illumination for the space defined by the imaging hood. Illumination from multiple light sources from different positions and angles provided by the plurality of light sources may facilitate imaging by preventing glare which may be caused by reflected light when visualizing illuminated tissue surfaces through the imaging hood. Moreover, illumination with this configuration may reduce shadow effects when tools are introduced into the hood.

### BRIEF DESCRIPTION OF THE DRAWING

FIG. 1A shows a side view of one variation of a tissue imaging apparatus during deployment from a sheath or delivery catheter.

FIG. 1B shows the deployed tissue imaging apparatus of FIG. 1A having an optionally expandable hood or sheath attached to an imaging and/or diagnostic catheter.

FIG. 1C shows an end view of a deployed imaging apparatus.

FIGS. 2A and 2B show one example of a deployed tissue imager positioned against or adjacent to the tissue to be imaged and a flow of fluid, such as saline, displacing blood from within the expandable hood.

FIGS. 3A and 3B show examples of various visualization imagers which may be utilized within or along the imaging hood.

FIGS. 4A and 4B show perspective and end views, respectively, of an imaging hood having at least one layer of a transparent elastomeric membrane over the distal opening of the hood.

FIGS. 5A and 5B show perspective and end views, respectively, of an imaging hood which includes a membrane with an aperture defined therethrough and a plurality of additional openings defined over the membrane surrounding the aperture.

FIGS. 6A and 6B show, respectively, a side view of the imaging hood visualizing a tissue region and the corresponding visual image resulting from axially-oriented visualization.

FIGS. 7A and 7B show for comparison, respectively, a side view of the imaging hood also visualizing, a tissue region and the corresponding visual image resulting from off-axis visualization provided via a solid state electronic imager.

FIG. 8A shows an assembly view of one variation of an imaging system coupled to an external imaging controller shown in a fragmentary perspective view.

FIG. 8B shows an assembly view of another variation where an imaging system is integrated within the visualization assembly.

FIG. 9A shows a detail perspective view of an imaging hood in its opened configuration with a solid state imager positioned interiorly along the inner portion of the hood which is off-axis relative to a longitudinal axis of the deployment catheter.

FIG. 9B shows a partial cross-sectional side view of an example illustrating the relative positioning of the off-axis solid state imaging system.

FIG. 10 shows an exploded perspective assembly view illustrating an example of the relative positioning of a lens assembly relative to an imaging sensor which may be square or circular in shape.

FIG. 11 shows an exploded perspective assembly view illustrating another example of an imaging system having a video processing board directly integrated with the imaging sensor.

FIG. 12 shows a schematic diagram illustrating the functional electronic components of the solid state imagers, e.g., CMOS imagers.

FIG. 13 shows an exploded perspective assembly view of another variation of an integrated imaging system interconnected via flexible pin connectors to increase flexibility of the imaging system.

FIG. 14 shows an exploded perspective assembly view of another variation of an integrated imaging system having its boards and sensor positioned and interconnected planarly.

FIGS. 15A and 15B show perspective and exploded perspective assembly views, respectively, of an integrated imaging system positioned planarly along an inner surface of the imaging hood and having one or more integrated lights, e.g., LED, thereon.

FIGS. 16A and 16B show end and exploded perspective assembly views, respectively, of an integrated imaging system positioned planarly along an inner surface of the imaging hood in a radial manner.

FIG. 17A shows a side view of a balloon visualization catheter integrated with a miniature solid state camera.

FIGS. 17B and 17C show a detail side view of imaging balloon and an exploded assembly view of the integrated imaging system, respectively.

FIGS. 18A and 18B show perspective and detail perspective views of a variation of a robotic control assembly showing a base having four proximal drive assemblies and an inflatable imaging balloon assembly positioned at a distal end of the catheter.

FIG. 18C illustrates an exploded assembly view of an integrated imaging system which may be incorporated into the robotically controlled imaging assembly.

### DETAILED DESCRIPTION OF THE INVENTION

A tissue-imaging and manipulation apparatus described herein is able to provide real-time images in vivo of tissue regions within a body lumen such as a heart, which is filled with blood flowing dynamically therethrough and is also able to provide intravascular tools and instruments for performing various procedures upon the imaged tissue regions. Such an apparatus may be utilized for many procedures, e.g., facilitating transeptal access to the left atrium, cannulating the coronary sinus, diagnosis of valve regurgitation/stenosis, valvuloplasty, atrial appendage closure, arrhythmogenic focus ablation, among other procedures.

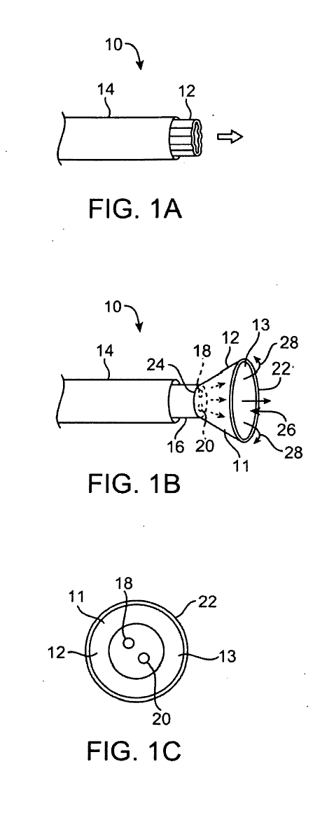

One variation of a tissue access and imaging apparatus is shown in the detail perspective views of FIGS. 1A to 1C. As shown in FIG. 1A, tissue imaging and manipulation assembly 10 may be delivered intravascularly through the patient's body in a low-profile configuration via a delivery catheter or sheath 14. In the case of treating tissue, it is generally desirable to enter or access the left atrium while minimizing trauma to the patient. To non-operatively effect such access, one conventional approach involves puncturing the intra-atrial septum from the right atrial chamber to the left atrial chamber in a procedure commonly called a transeptal procedure or septostomy. For procedures such as percutaneous valve repair and replacement, transeptal access to the left atrial chamber of the heart may allow for larger devices to be introduced into the venous system than can generally be introduced percutaneously into the arterial system.

When the imaging and manipulation assembly 10 is ready to be utilized for imaging tissue, imaging hood 12 may be advanced relative to catheter 14 and deployed from a distal opening of catheter 14, as shown by the arrow. Upon deployment, imaging hood 12 may be unconstrained to expand or open into a deployed imaging configuration, as shown in FIG. 1B. Imaging hood 12 may be fabricated from a variety of pliable or conformable biocompatible material including but not limited to, e.g., polymeric, plastic, or woven materials. One example of a woven material is Kevlar® (E. I. du Pont de Nemours, Wilmington, Del.), which is an aramid and which can be made into thin, e.g., less than 0.001 in., materials which maintain enough integrity for such applications described herein. Moreover, the imaging hood 12 may be fabricated from a translucent or opaque material and in a variety of different colors to optimize or attenuate any reflected lighting from surrounding fluids or structures, i.e., anatomical or mechanical structures or instruments. In either case, imaging hood 12 may be fabricated into a uniform structure or a scaffold-supported structure, in which case a scaffold made of a shape memory alloy, such as Nitinol, or a spring steel, or plastic, etc., may be fabricated and covered with the polymeric, plastic, or woven material. Hence, imaging hood 12 may comprise any of a wide variety of barriers or membrane structures, as may generally be used to localize displacement of blood or the like from a selected volume of a body lumen or heart chamber. In exemplary embodiments, a volume within an inner surface 13 of imaging hood 12 will be significantly less than a volume of the hood 12 between inner surface 13 and Outer surface 11.

Imaging hood 12 may be attached at interface 24 to a deployment catheter 16 which may be translated independently of deployment catheter or sheath 14. Attachment of interface 24 may be accomplished through any number of conventional methods. Deployment catheter 16 may define a fluid delivery lumen 18 as well as an imaging lumen 20 within which an optical imaging fiber or assembly may be disposed for imaging tissue. When deployed, imaging hood 12 may expand into any number of shapes, e.g., cylindrical, conical as shown, semi-spherical, etc., provided that an open area or field 26 is defined by imaging hood 12. The open area 26 is the area within which the tissue region of interest may be imaged. Imaging hood 12 may also define an atraumatic contact lip or edge 22 for placement or abutment against the tissue region of interest. Moreover, the diameter of imaging hood 12 at its maximum fully deployed diameter, e.g., at contact lip or edge 22, is typically greater relative to a diameter of the deployment catheter 16 (although a diameter of contact lip or edge 22 may be made to have a smaller or equal diameter of deployment catheter 16). For instance, the contact edge diameter may range anywhere from 1 to 5 times (or even greater, as practicable) a diameter of deployment catheter 16. FIG. 1C shows an end view of the imaging hood 12 in its deployed configuration. Also shown are the contact lip or edge 22 and fluid delivery lumen 18 and imaging lumen 20.

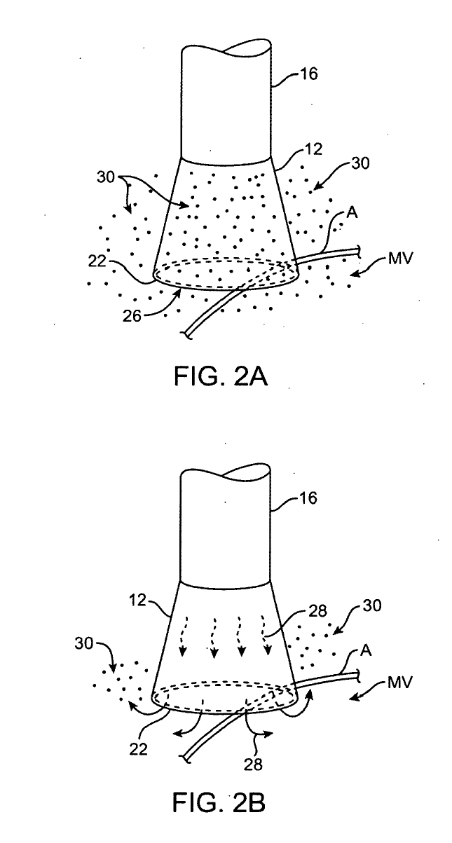

As seen in the example of FIGS. 2A and 2B, deployment catheter 16 may be manipulated to position deployed imaging hood 12 against or near the underlying tissue region of interest to be imaged, in this example a portion of annulus A of mitral valve MV within the left atrial chamber. As the surrounding blood 30 flows around imaging hood 12 and within open area 26 defined within imaging hood 12, as seen in FIG. 2A, the underlying annulus A is obstructed by the opaque blood 30 and is difficult to view through the imaging lumen 20. The translucent fluid 28, such as saline, may then be pumped through fluid delivery lumen 18, intermittently or continuously, until the blood 30 is at least partially; and preferably completely, displaced from within open area 26 by fluid 28, as shown in FIG. 2B.

Although contact edge 22 need not directly contact the underlying tissue, it is at least preferably brought into close proximity to the tissue such that the flow of clear fluid 28 from open area 26 may be maintained to inhibit significant backflow of blood 30 back into open area 26. Contact edge 22 may also be made of a soft elastomeric material such as certain soft grades of silicone or polyurethane, as typically known, to help contact edge 22 conform to an uneven or rough underlying anatomical tissue surface. Once the blood 30 has been displaced from imaging hood 12, an image may then be viewed of the underlying tissue through the clear fluid 30. This image may then be recorded or available for real-time viewing for performing a therapeutic procedure: The positive flow of fluid 28 may be maintained continuously to provide for clear viewing of the underlying tissue. Alternatively, the fluid 28 may be pumped temporarily or sporadically only until a clear view of the tissue is available to be imaged and recorded, at which point the fluid flow 28 may cease and blood 30 may be allowed to seep or flow back into imaging hood 12. This process may be repeated a number of times at the same tissue region or at multiple tissue regions.

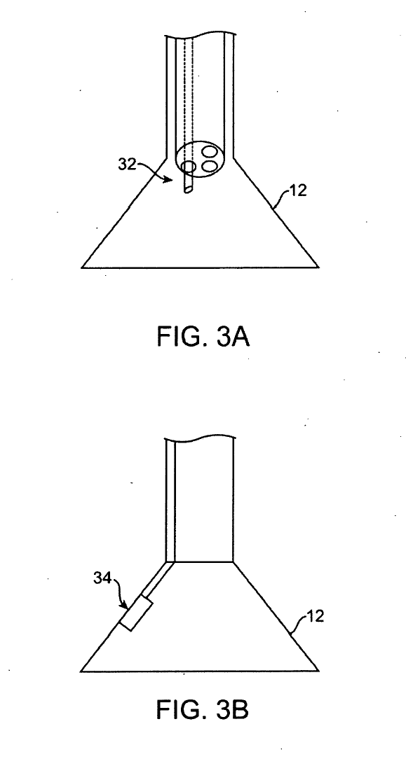

FIG. 3A shows a partial cross-sectional view of an example where one or more optical fiber bundles 32 may be positioned within the catheter and within imaging hood 12 to provide direct in-line imaging of the open area within hood 12. FIG. 3B shows another example where an imaging element 34 (e.g., CCD or CMOS electronic imager) may be placed along an interior surface of imaging hood 12 to provide imaging of the open area such that the imaging element 34 is off-axis relative to a longitudinal axis of the hood 12, as described in further detail below. The off-axis position of element 34 may provide for direct visualization and uninhibited access by instruments from the catheter to the underlying tissue during treatment.

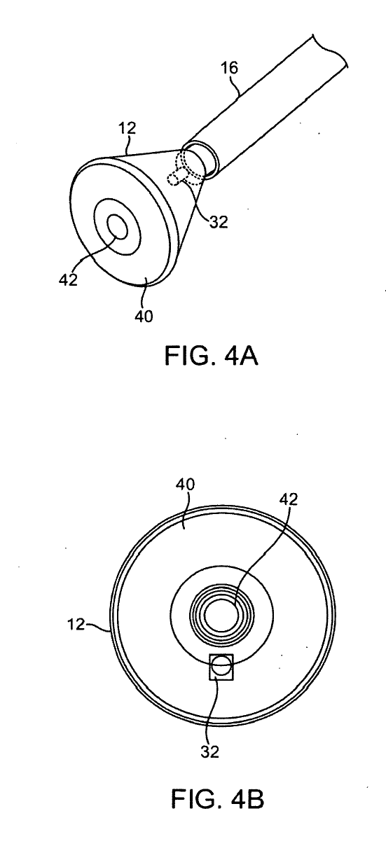

In utilizing the imaging hood 12 in any one of the procedures described herein, the hood 12 may have an open field which is uncovered and clear to provide direct tissue contact between the hood interior and the underlying tissue to effect any number of treatments upon the tissue, as described above. Yet in additional variations, imaging hood 12 may utilize other configurations. An additional variation of the imaging hood 12 is shown in the perspective and end views, respectively, of FIGS. 4A and 4B, where imaging hood 12 includes at least one layer of a transparent elastomeric membrane 40 over the distal opening of hood 12. An aperture 42 having a diameter which is less than a diameter of the outer lip of imaging hood 12 may be defined over the center of membrane 40 where a longitudinal axis of the hood intersects the membrane such that the interior of hood 12 remains open and in fluid communication with the environment external to hood 12. Furthermore, aperture 42 may be sized, e.g., between 1 to 2 mm or more in diameter and membrane 40 can be made from any number of transparent elastomers such as silicone, polyurethane, latex, etc. such that contacted tissue may also be visualized through membrane 40 as well as through aperture 42.

Aperture 42 may function generally as a restricting passageway to reduce the rate of fluid out-flow from the hood 12 when the interior of the hood 12 is infused with the clear fluid through which underlying tissue regions may be visualized. Aside from restricting out-flow of clear fluid from within hood 12, aperture 42 may also restrict external surrounding fluids from entering hood 12 too rapidly. The reduction in the rate of fluid out-flow from the hood and blood in-flow into the hood may improve visualization conditions as hood 12 may be more readily filled with transparent fluid rather than being filled by opaque blood which may obstruct direct visualization by the visualization instruments.

Moreover, aperture 42 may be aligned with catheter 16 such that any instruments (e.g., piercing instruments, guidewires, tissue engagers, etc.) that are advanced into the hood interior may directly access the underlying tissue uninhibited or unrestricted for treatment through aperture 42. In other variations wherein aperture 42 may not be aligned with catheter 16, instruments passed through catheter 16 may still access the underlying tissue by simply piercing through membrane 40.

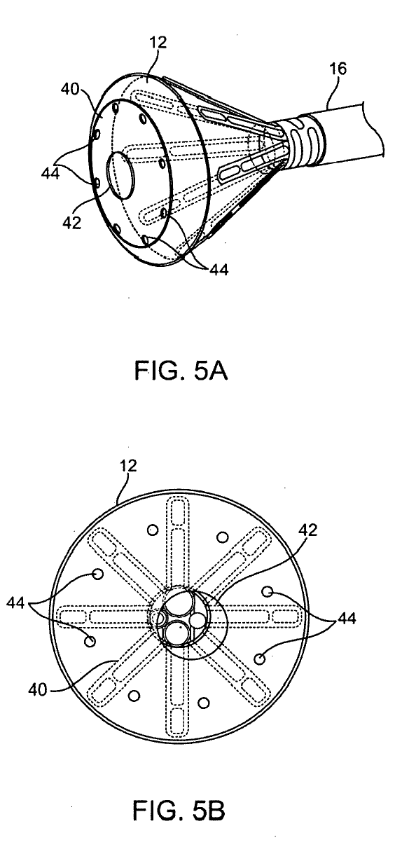

In an additional variation, FIGS. 5A and 5B show perspective and end views, respectively, of imaging hood 12 which includes membrane 40 with aperture 42 defined therethrough, as described above. This variation includes a plurality of additional openings 44 defined over membrane 40 surrounding aperture 42. Additional openings 44 may be uniformly sized, e.g., each less than 1 mm in diameter, to allow for the out-flow of the translucent fluid therethrough when in contact against the tissue surface. Moreover, although openings 44 are illustrated as uniform in size, the openings may be varied in size and their placement may also be non-uniform or random over membrane 40 rather than uniformly positioned about aperture 42 in FIG. 5B. Furthermore, there are eight openings 44 shown in the figures although fewer than eight or more than eight openings 44 may also be utilized over membrane 40.

Additional details of tissue imagine and manipulation systems and methods which may be utilized with apparatus and methods described herein are further described, for example, in U.S. patent application Ser. No. 11/259,498 filed Oct. 25, 2005 (U.S. Pat. Pub. No. 2006/0184048 A1); Ser. No. 11/763,399 filed Jun. 14, 2007 (U.S. Pat. Pub. No. 2007/0293724 A1); and also in Ser. No. 11/828,267 filed Jul. 25, 2007 (U.S. Pat. Pub. No. 2008/0033290 A1), each of which is incorporated herein by reference in its entirety.

In imaging the underlying tissue for diagnosis and/or treatment, optical fiberscopes may be positioned within the deployment catheter to view the tissue from an axial orientation in-line with a longitudinal axis of the catheter and/or imaging hood. Alternatively, other variations may utilize high resolution imaging systems integrated into the imaging hood. Such imaging systems can include solid state cameras utilizing CMOS (complementary metal oxide semi-conductors) or CCD (charged couple devices) technology where elements of the imaging device may be separated from one another and interconnected by pin connectors or cables such that the imaging assembly may be mounted within or along an inner surface of the imaging hood. Such configurations may also allow the solid state cameras to be compacted into a relatively small volumes with low profiles and/or a flexible configurations.

Generally, such a solid state electronic imaging system may comprise in one variation a lens assembly, an electronic imaging sensor positioned adjacent to the lens assembly for receiving an image from the lens assembly, and a video processing assembly electrically coupled to the imaging sensor via a flexible connector where the imaging system is sized to be positioned within or along an inner surface of an imaging hood which is reconfigurable between a delivery profile and a deployment profile. In use, the imaging assembly may be intravascularly advanced by a deployment catheter to the tissue region of interest where the imaging assembly may be reconfigured from a delivery profile to a deployment profile such that the imaging assembly defines an open area in fluid communication with a fluid lumen defined through the catheter. Once reconfigured into the deployed profile, the open area of the device may be positioned against the tissue region such that blood is displaced from the open area and the tissue may then be visualized, e.g., via the off-axis electronic imaging assembly positioned within or along an inner surface of the imaging assembly.

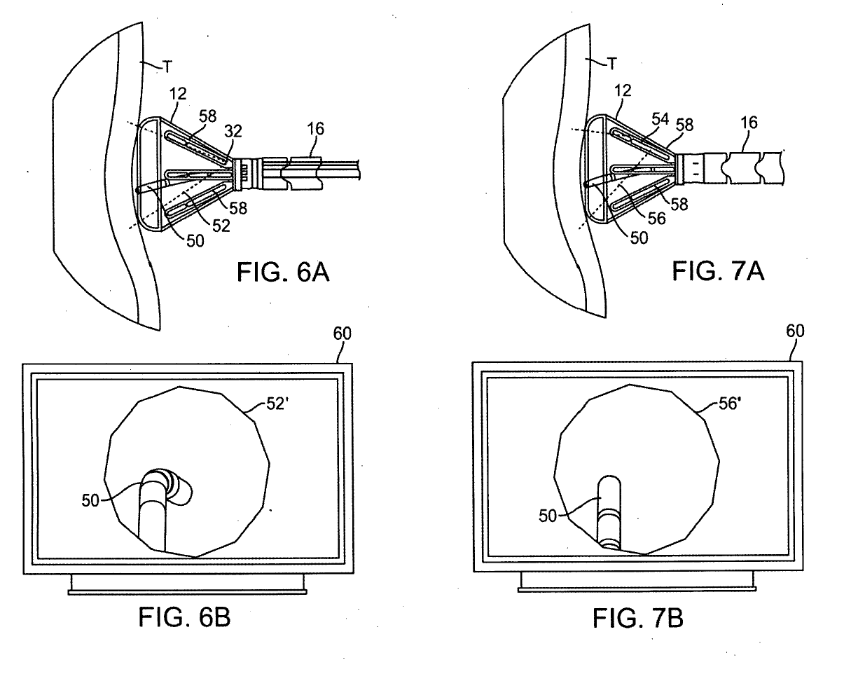

As aforementioned, when tissue is visualized via a fiberscope, the field of view 52 provided by the fiberscope is limited to an axially oriented image relative to the longitudinal axis of the catheter 16, as illustrated in the side view of FIG. 6A. The corresponding axially-oriented image 52′ is representatively displayed on monitor 60 in FIG. 6B, which illustrates the tissue region obstructed by instrument 50, e.g., ablation probe, which may be positioned within the open field defined by hood 12. In comparison, all electronic imaging element 54, e.g., CMOS imager or camera, positioned off-axis along one or more support struts 58 within or along the inner surface of hood 12 may provide an angled field of view 56 from CMOS imager 54, as shown in the side view of FIG. 7A. The corresponding off-axis image 56′ is represented on the monitor 60 in FIG. 7B. As compared to in-line visualization along the axis of the catheter, off-axis visualization may be particularly advantageous in allowing operators to view and gauge distances between deployed tools and tissue. Off-axis visualization may also be particularly advantageous in providing a better visual confirmation of contact between instruments and tissue. In therapies such as atrial fibrillation ablation or ventricular tachycardia ablation where contact between the ablation probe and tissue is desirable in the formation of effective lesions, off-axis visualization may facilitate the treatment and efficacy of such endocardial therapies.

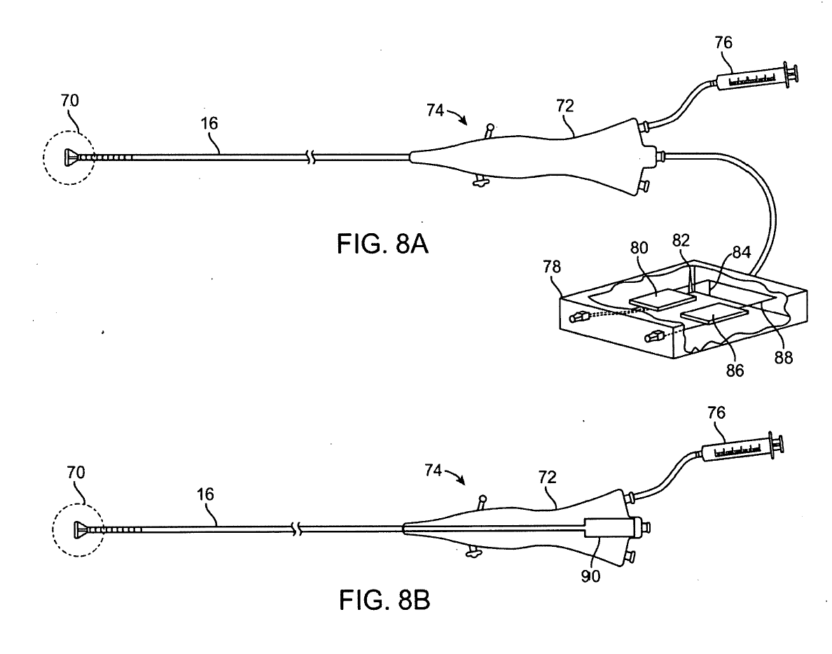

In utilizing the electronic solid state imaging element, e.g., CMOS imager or camera, the imaging assembly positioned within the visualization and/or treatment assembly 70 may be electrically coupled to control assembly 78 through deployment catheter 16 and handle 72. The variation illustrated in the assembly of FIG. 8A shows handle 72 coupled to control assembly 78 through a cable and which may remain external to the patient body during a procedure. Fluid reservoir 76, e.g., syringe, and articulation and manipulation controls 74 are illustrated coupled to handle 72 as well.

In this variation, the lens assembly and image sensors of the electronic imaging assembly may be located within visualization and/or treatment assembly 70 while separated from the remaining circuitry. The remaining circuitry may include a video processing board 86 and power supply board 80 that can be housed in control assembly 78. The power supply board 80 may be electrically coupled to the rest of the imaging assembly via power conductor 82 while the video processing board 86 may be coupled via image signal conductor 88. Grounding conductor 84 may also be coupled within control assembly 78. The control assembly 78 can communicate with the miniature CMOS camera in the imaging hood 12 via connection cables coupling the CMOS camera, through the work channel of catheter 16, through handle 72, and into the control assembly 78.

FIG. 8B shows a side view of another variation where the imaging system may be self-contained entirely within the visualization and/or treatment assembly 70, as described in detail below. Control assembly 78 may be replaced with an integrated power supply 90, e.g., a battery, that is housed within handle 72 such that the entire imaging system is integrated within the visualization catheter system. Direct current may be supplied by the integrated power supply 90 such that the power supplied to the solid state image sensors may range, e.g., between 1.5 V to 12 V.

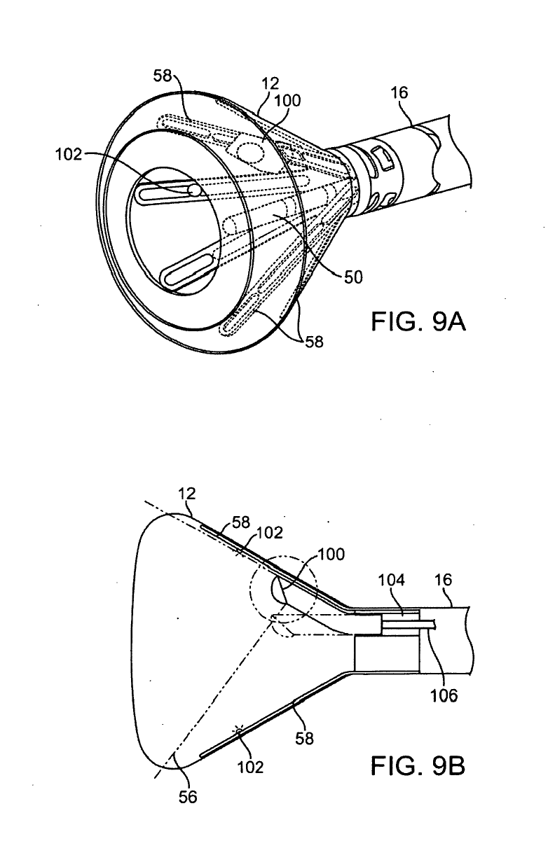

FIG. 9A shows a detailed perspective view of imaging hood 12 with the housing of solid state imager assembly 100, e.g., CMOS camera mounted off-axis to the longitudinal axis of the catheter, along the inner proximal wall and/or supporting struts 58 of hood 12. This variation utilizes an integrated imaging system where the image sensor, processor and other circuitry is fully contained and integrated within the housing of imager assembly 100. One or more light sources 102, e.g., light emitting diodes (LEDs), may be mounted around the interior of the imaging hood 12 along one or more struts 58 to provide illumination for the space defined by imaging hood 12. Illumination from multiple light sources 102 located within hood 12 at different positions and angles relative to one another may prevent glare caused by excessive reflected light when visualizing illuminated tissue surfaces through the imaging hood 12. Additionally, illumination with this configuration may reduce any shadow effects when instruments are introduced into hood 12. Additionally, the side walls of hood 12 may also be made with opaque polymers, such as barium sulfate, or coated with a reflective coating to prevent loss of light from the hood 12. Such features may decrease the number of light sources 102 used to provide the same level of illumination on visualized tissue surfaces.

FIG. 9B illustrates a partial cross-sectional side view of a variation where solid state imager assembly 100 may be positioned along the inner surface of hood 12. The electrical conductors or wires 106 coupling the imaging assembly to the power supply located within handle 72 and to the monitor for viewing may be passed through lumen 104 defined through deployment catheter 16.

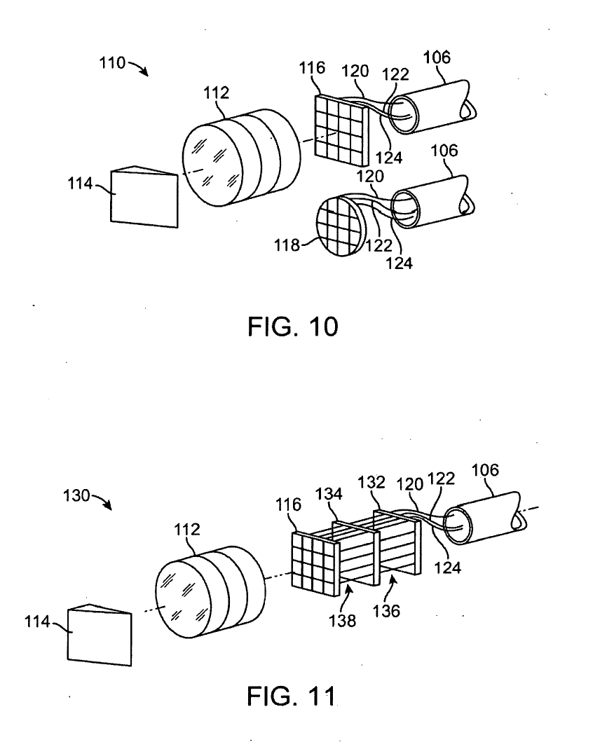

An example illustrating the relative positioning within CMOS imaging assembly 110 mounted along the inner surface of imaging hood 12 is shown in the exploded perspective assembly view of FIG. 10. The housing of imaging assembly 110 has been omitted for clarity. As shown, because the imagine assembly 110 may be positioned along hood 12 at an angle relative to the catheter 16 and tissue surface being visualized, imaging assembly 110 may include an optional prism 114 positioned distal to lens assembly 112 for capturing images from its off-axis position. Lens assembly 112 may be positioned adjacent to an imaging sensor which may be configured into a square imaging sensor 116 or alternatively into a circular imaging sensor 118 depending upon the housing configuration, each variation illustrated as alternative configurations.

In either case, the images captured through optional prism 114 and lens assembly 112 may be projected onto the imaging sensor 116 or 118 which may convert the images to electrical signals for transmission through image signal conductor 124 back to the control assembly. Power may be supplied to the image sensor through power conductor 120 and the image sensor may also be connected to grounding conductor 122. With the image sensors ranging in size as small as 1 mm in diameter and by further separating elements of the CMOS imaging system 110 and integrating these separate elements into the visualization catheter, a compact imaging element can be mounted onto the imaging hood 12 without the need to compromise on image resolution normally available through the use of imaging modalities such as fiberoptics.

In another variation, FIG. 11 shows an exploded perspective assembly view illustrating an example of an integrated solid state imaging system 130 having a video processing board 132 directly integrated with the imaging sensor 116. Video processing board 132 may be placed directly proximal to the image sensor 116 in the imaging element assembly 130 found in the imaging hood 12. A plurality of pin connectors 136, 138 can be connected between the image sensor 116 and the video processing board 132 to electrically couple the image sensor 116 to the video processing board 132 or to provide a conduction path through which image signals may be transmitted between image sensor 116 and the video processing board 132. If necessary or desirable, one or more supplementary boards 134 may be connected to the video processing board 132 using pin connectors 136, 138 to further contain processing circuitry for processing the image signals which may be directly received by a desired video device. Hence in this variation, by stacking the CMOS imaging circuitry adjacent to one another in a parallel configuration and integrating such circuitry into the tissue visualization catheter, a compact yet high-resolution imaging element can be placed into the imaging hood 12.

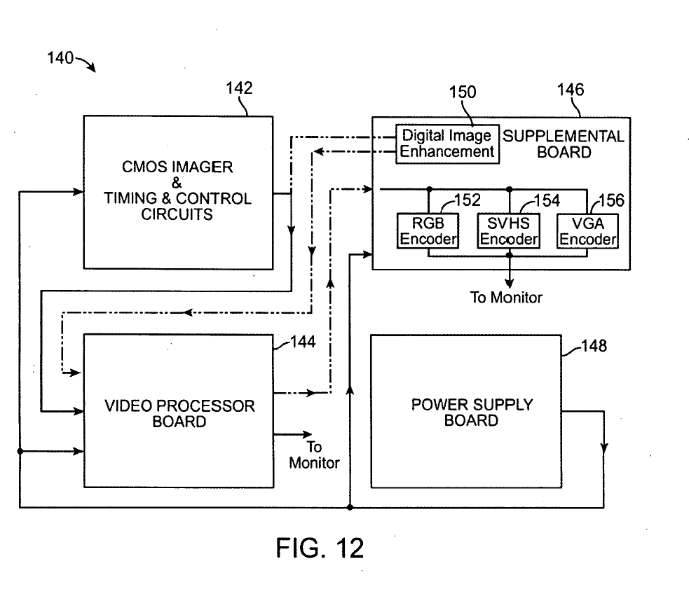

FIG. 12 shows a schematic diagram 140 of the functional electronic components which may be included in an electronic imaging system. As shown, CMOS imager 142 and timing and control circuits may receive the images for transmission to video processor board 144 which may be coupled to a monitor for display and which may also in turn be optionally coupled to a supplemental board 146, which may include digital image enhancement 150 circuitry as well as one or more encoders, e.g., RGB encoder 152, SVHS encoder 154, VGA encoder 156, etc. The power supply board 148 may be electrically coupled to each of the components to provide power. The functional electronic components illustrated may be encompassed by either configuration where the external control assembly is utilized or where the complete imaging system is fully integrated within the visualization catheter. Further details of the circuitry and their functional electronic components which may be utilized herein may be found in U.S. Pat. No. 5,929,901 which is incorporated herein by reference in its entirety.

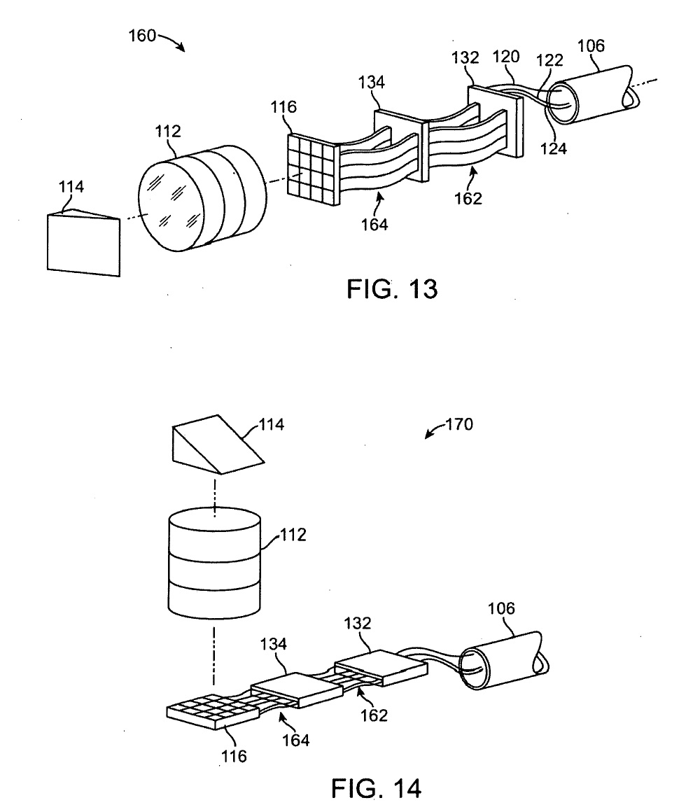

FIG. 13 illustrates an exploded perspective view of another variation of a miniature integrated CMOS imaging system 160 which is flexible and bendable along its longitudinal axis. Each of the components, e.g., image sensor 116, video processor board 132, one or more optional supplementary boards 134, etc., may be coupled via flexible pin connectors 162, 164 such that each component may be translated and/or moved off-axis relative to one another from its original plane when the imaging system 160 is subjected to axial or bending forces thus allowing imaging system 160 to be flexible. The flexible imaging system may be insulated with a flexible casing made from medical grade polyurethane, silicone, or other elastomers. In this configuration, the bendable miniature imaging system 160 may be able to accommodate hood movements during delivery or deployment when hood 12 is deployed from its collapsed configuration to its expanded configuration. A miniature imaging system 160 that is bendable may also facilitate the delivery of the visualization catheter into a patient's body when the catheter 16 is subjected to tortuous vasculature.

Yet another variation is illustrated in the exploded perspective assembly view of integrated CMOS imaging assembly 170 in FIG. 14. In this variation, the imaging sensor 116, supplementary board 134, and the video processing board 132 may be arranged in a co-linear and planar configuration where the flexible pin connectors 162, 164 may be attached between each component along the edges of each board. The lens assembly 112 and optional prism 114 may be mounted atop the imaging sensor 116. Such an imaging assembly 170 may be integrated into the visualization catheter by mounting along the longitudinal axis of one of the struts 58 of imaging hood 12. Alternatively, imaging assembly 170 may be integrated within the wall of hood 12 to also function structurally as a strut by having the circuit boards coated with the polymer such as Chronoflex®. Utilizing a co-linear and planar configuration may enable the separation of elements of the imaging system into different segments and aligning them adjacent to each other within the same plane such that the system forms a flattened and flexible assembly may allow an imaging hood 12 to be easily collapsed and/or retractable into a sheath.

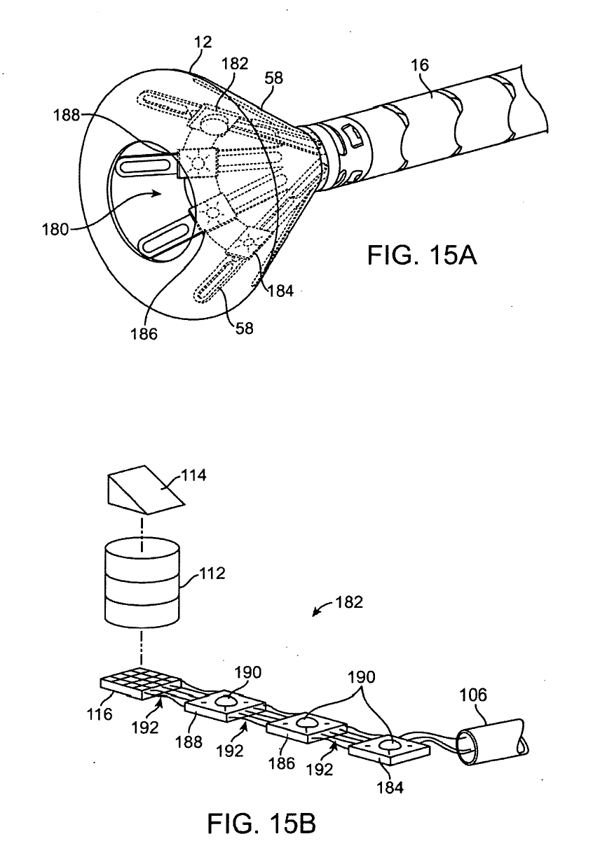

FIGS. 15A and 15B show yet another variation of an integrated CMOS imaging system 180 arranged in arcuate or curved orientation within or along the interior surface of imaging hood 12. In this configuration, each of the components comprising imaging assembly 182 (e.g., imaging sensor 116, supplementary board(s) 186, 188, video processing board 184, etc.) can each be individually mounted on a support strut 58 of imaging hood 12. The flexible pin connectors 192 can be used to connect the boards such that the entire imaging system 180 is circumferentially arranged along the inner wall of the imaging hood 12. Optionally, one or more light sources 190, e.g., LEDs; can be mounted to and powered by one or more of the component boards such that illumination within the open area of the hood 12 is directed from multiple angles to prevent glare or shadow effects.

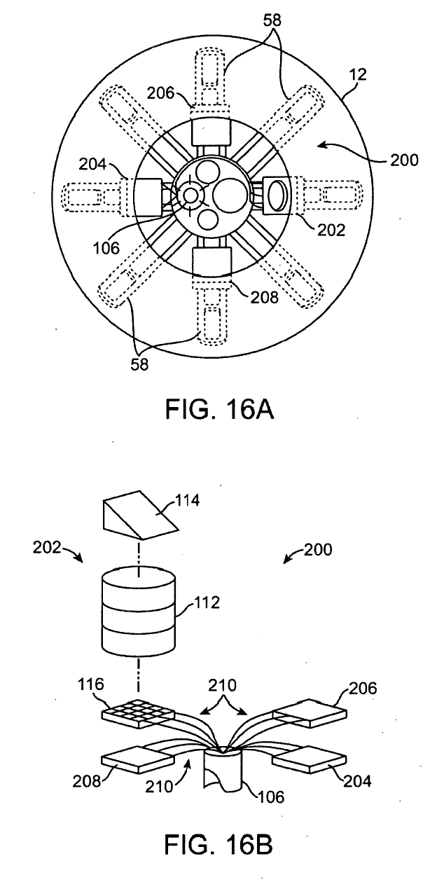

FIGS. 16A and 16B show end and exploded perspective assembly views, respectively, of yet another variation of an integrated CMOS imaging assembly 200 positioned planarly along an inner surface of the imaging hood 12 in a radial manner. Each of the components comprising imaging assembly 202 (e.g., imaging sensor 116, supplementary board(s) 206, 208, video processing board 204, etc.) can each be connected via flexible pin connectors 210 along each side of a board towards a central focal point where the connection pins may be coupled accordingly to interact with other circuitry boards. As described above, each component may be supported or mounted along the inner surface by the one or more support struts 58.

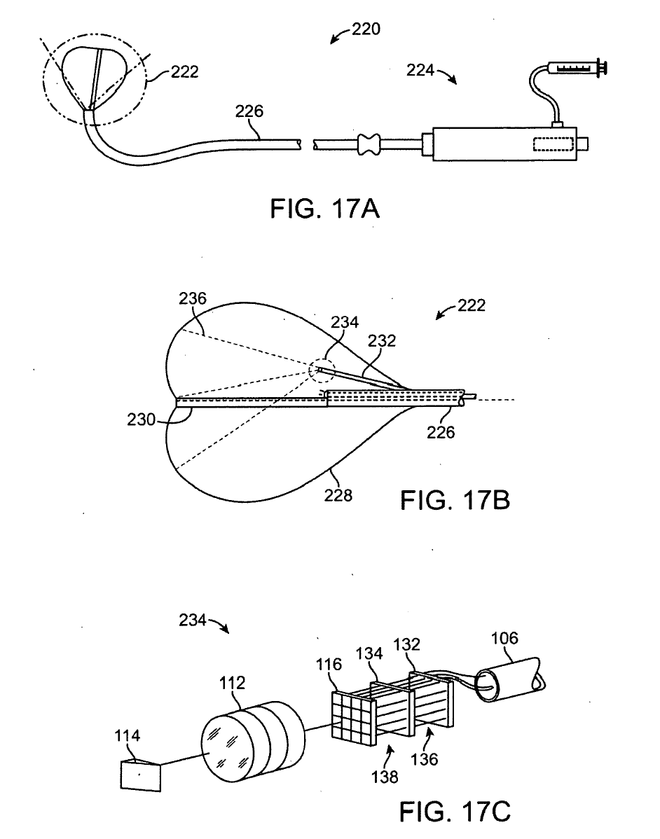

In yet another variation, FIGS. 17A and 17B show a detail side view of an imaging balloon and an exploded assembly view of the integrated imaging system, respectively. In this particular variation, rather than utilizing an open hood or a hood having an aperture in fluid communication with the environment, inflatable balloon imaging system 220 utilizes visualization assembly 222 having an inflatable transparent balloon member 228 disposed on a catheter 226 extending from handle assembly 224. A working lumen 230 extends through the catheter and balloon member 228 and an off-axis imaging arm 232, which may be angled relative to the catheter 226, may include an imaging assembly 234 integrated on its distal tip. The imaging assembly 234 from its off-axis position may provide a field of view 236 about the distal end of the balloon member 228. As previously described, the imaging assembly 234 may integrate a system, as shown in the perspective view of FIG. 17C, where the lens assembly 112, imaging sensor 116, video processing board 132, and optional supplementary boards 134 may be interconnected via pin connectors 136, 138 to form an imaging assembly 234 contained entirely within the inflatable balloon member 228. Further details of the balloon member and other variations thereof are described in detail in U.S. Pat. No. 6,979,290 B2, which is incorporated herein by reference in its entirety.

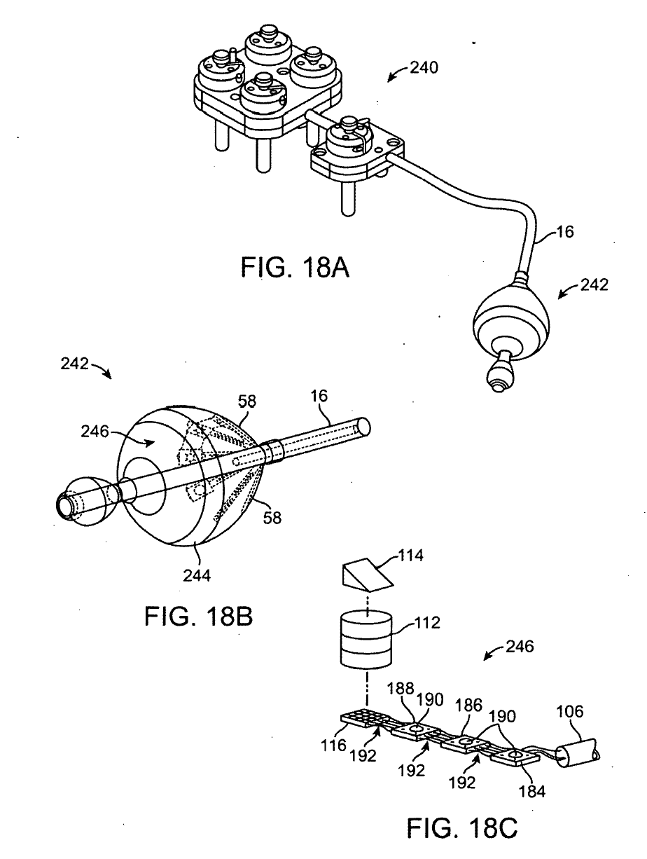

The imaging system described may also be incorporated into other catheter control systems, for example, as shown in the perspective views of FIGS. 18A and 18B. FIG. 18A illustrates a perspective view of a variation of a robotic control assembly which may be articulated via control assembly 240 which articulates a deployment catheter 16 having an inflatable imaging balloon 242 assembly attached thereto. Precise movements of the distal tip of the catheter 16 can be accordingly controlled by robotic mechanisms. Imaging balloon 244 may be inflated with a transparent fluid or gas, as described above, and may be further supported structurally by one or more support struts 58 extending distally from catheter 16 within or along a proximal portion of imaging balloon 244. During introduction and/or advancement through the patient body, support struts 58 may be collapsed along with imaging balloon 244 into a low-profile configuration and when deployed, balloon 244 may be inflated and support struts 58 may extend radially relative to catheter 16 to support imaging balloon 244. Additionally, support struts 58 may also support imaging assembly 246, as described above and as shown in the exploded perspective view of FIG. 18C, along or upon one or more support struts 58 for viewing the tissue region through balloon 244 as well. Additional variations and details of the various catheter control systems which may incorporate an integrated imaging system are described in U.S. Pat. Pub. No. 2006/0084945 A1 as well as U.S. patent application Ser. No. 11/848,429 filed August 31, 2007 (U.S. Pat. Pub. No. 2008/0097476 A1), each of which is incorporated herein by reference in its entirety.

Additionally, such planarly configured and flexible CMOS imaging systems may be utilized in other catheter systems such as those described in U.S. Pat. Pub. No. 2004/0006333 A1, which is also incorporated herein by reference in their entirety. The visualization balloon can be transparent to visualize tissue through opaque fluids such as blood and can be at least partially transmissive to ablation energy, such as laser energy.

The applications of the disclosed invention discussed above are not limited to certain treatments or regions of the body, but may include any number of other treatments and areas of the body. Modification of the above-described methods and devices for carrying out the invention, and variations of aspects of the invention that are obvious to those of skill in the arts are intended to be within the scope of this disclosure. Moreover, various combinations of aspects between examples are also contemplated and are considered to be within the scope of this disclosure as well.

What is claimed is:

1. A solid state electronic imaging system, comprising:
a lens assembly;
an electronic imaging sensor positioned adjacent to the lens assembly for receiving an image from the lens assembly; and
a video processing assembly electrically coupled to the imaging sensor via a flexible connector,
wherein the imaging system is sized to be positioned within or along an inner surface of an imaging hood which is reconfigurable between a delivery profile and a deployment profile.

2. The system of claim 1 further comprising a prism positioned distal to the lens assembly.

3. The system of claim 1 wherein the imaging sensor and video processing assembly are aligned in parallel with the lens assembly.

4. The system of claim 1 wherein the imaging sensor and video processing assembly are planarly aligned with respect to one another.

5. The system of claim 1 further comprising at least one supplemental board assembly interconnected with the imaging sensor and/or video processing assembly.

6. The system of claim 1 further comprising at least one light source integrated with the imaging system.

7. The system of claim 6 wherein the at least one light source comprises a light emitting diode.

8. The system of claim 1 wherein the imaging sensor and video processing assembly are coupled in series.

9. The system of claim 1 wherein the imaging sensor and video processing assembly are positioned in series along the inner surface of the imaging hood.

10. The system of claim 1 wherein the imaging sensor and video processing assembly are positioned radially along the inner surface of the imaging hood.

11. A tissue visualization system, comprising:
a deployment catheter having a distal end;
an imaging assembly extending from the distal end and defining an inner surface and an outer surface and which is configurable between a delivery profile and a deployment profile, wherein the imaging assembly defines an open area in fluid communication with a fluid lumen defined through the catheter;
an electronic imaging assembly positioned within or along the inner surface of the imaging assembly such that a tissue region defined by the open area is imaged by the imaging assembly, and
wherein the imaging assembly comprises an electronic imaging sensor and a video processing assembly coupled to the imaging sensor via a flexible connector such that the imaging assembly is flexibly configured along the inner surface.

12. The system of claim 11 wherein the deployment catheter is articulatable.

13. The system of claim 12 wherein the deployment catheter is robotically controlled.

14. The system of claim 11 wherein the imaging assembly comprises a hood defining at least one aperture through a distal membrane over the open area

15. The system of claim 11 wherein the imaging assembly comprises an inflatable balloon member.

16. The system of claim 11 further comprising at least one supplemental board assembly interconnected with the imaging sensor and/or video processing assembly.

17. The system of claim 11 further comprising at least one light source integrated with the imaging assembly.

18. The system of claim 17 wherein the at least one light source comprises a light emitting diode.

19. The system of claim 11 wherein the imaging sensor and video processing assembly are coupled in series.

20. The system of claim 11 wherein the imaging sensor and video processing assembly are positioned in series along the inner surface.

21. The system of claim 1 wherein the imaging sensor and video processing assembly are positioned radially along the inner surface.

22. A method of visualizing a tissue region, comprising:
intravascularly advancing an imaging assembly extending from a distal end of a deployment catheter to a tissue region of interest;
reconfiguring the imaging assembly from a delivery profile to a deployment profile such that the imaging assembly defines an open area in fluid communication with a fluid lumen defined through the catheter;
positioning the open area against the tissue region of interest such that blood is displaced from the open area; and
visualizing the tissue region of interest via an off-axis electronic imaging assembly positioned within or along an inner surface of the imaging assembly, wherein the imaging assembly comprises an electronic imaging sensor and a video processing assembly coupled to the imaging sensor via a flexible connector such that the imaging assembly is flexibly configured along the inner surface.

23. The method of claim 22 wherein intravascularly advancing comprises articulating the imaging assembly via the catheter.

24. The method of claim 23 wherein articulating comprises robotically articulating the imaging assembly.

25. The method of claim 22 wherein reconfiguring comprises configuring an imaging hood from a delivery configuration to a deployment configuration.

26. The method of claim 22 wherein reconfiguring comprises inflating an expandable balloon member from a delivery configuration to an expanded deployment configuration.

27. The method of claim 22 wherein positioning comprises displacing the blood between an expanded balloon and the tissue region of interest.

28. The method of claim 22 wherein positioning comprises displacing the blood from the open area via a transparent fluid introduced through the fluid lumen.

29. The method of claim 22 wherein the imaging assembly is reconfigured from a collapsed profile to an expanded profile upon reconfiguring the imaging assembly.
